(19) United States
(12) Patent Application Publication
(10) Pub. No.: US 2002/0147495 A1
(43) Pub. Date: Oct. 10, 2002

(54) Reduced-size replacement heart

(76) Inventor: Christopher Petroff, Revere, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 09/829,404

(22) Filed: Apr. 9, 2001

(51) Int. Cl. A61M 1/12

(52) U.S. Cl. 623/3.25; 623/3.28; 600/16

(57) ABSTRACT

An electrohydraulic energy converter useful in a circulatory assist device includes a housing defining a hydraulic fluid flow path and having first and second open ends. A left diaphragm is disposed on and seals the first open end of the housing, and a right diaphragm is disposed on and seals the second open end of the housing. A reversible axial flow pump is disposed within the housing for reversibly pumping hydraulic fluid along the hydraulic fluid flow path. In another embodiment, a blood pump is provided for alternately driving left and right systole in a circulatory assist device. The blood pump includes an energy converter having left and right blood pumping elements disposed on and sealing the two open ends of the housing. In a further embodiment, the axial flow pump includes a stator, a rotor rotatably connected to the stator, several impeller blades disposed around and extending radially outward from the rotor, and a motor for imparting mechanical movement on the rotor. The impeller blades act against fluid to pump the fluid to and from the left and right blood pumping elements or diaphragms. The stator may contain several stator blades positioned on opposed sides of the impeller blades along the fluid flow path.

### BACKGROUND OF THE INVENTION

The present invention relates to fluid pumps and more particularly to specialized pumping assemblies for use in or as circulatory assist devices, including blood pumps, ventricular assist devices, and artificial hearts.

Several types of surgically implantable pumps have been developed in an effort to augment or replace the blood pumping action of damaged or diseased hearts. Common types of pumps include positive displacement pumps such as piston-type pumps, moving diaphragm pumps, peristaltic action pumps, centrifugal pumps, and the like.

Rotary pumps are often the preferred type of pumps for use in circulatory assist devices. Rotary pumping mechanisms can be classified into two categories: centrifugal pumps and axial pumps. Centrifugal pumps include a pump housing, an inlet aligned with a rotational axis of the pump, and an outlet around the periphery of the housing. The pumping action derives from the rotation of an impeller within the housing that draws the fluid in axially, and then expels it outward away from the rotational axis of the pump. Axial pumps provide fluid inflow and outflow along a straight line along the direction of the rotational axis of the pump. In an axial flow pump, impeller blades are mounted on a rotor mounted inside a housing that provides for axial flow through the pump. As the blade assembly spins, the impeller blades impart a force onto fluid to drive axial fluid flow through the housing.

Rotary pumps can be used to displace blood directly, or they may be used within electrohydraulic energy converters that pump hydraulic fluid to move one or more diaphragms. The movable diaphragms can then drive one or more pumping volumes that can be filled with blood that is subsequently expelled in a manner similar to the operation of a natural ventricle. One such design for an electrohydraulic drive system is set forth in U.S. Pat. No. 4,173,796 by Jarvik. The Jarvik energy converter system employs an axial flow pump to convert electric energy from a power source into hydraulic power capable of actuating a diaphragm, sack, or other collapsible volume blood pump drive device. The theory of this device was to place the motor at the base of a total artificial heart and reversibly transfer hydraulic pumping fluid between respective pumping chambers of artificial left and right ventricles. The motor is situated below the blood chambers and is connected to the chambers using hydraulic fluid flow channels.

There are a number of problems associated with the Jarvik design. Response time of the pumping chambers to the axial flow pump is not optimal due to the channel configuration, which imposes a substantial distance of travel for the fluid as it is moves between the axial flow pump and the pumping chambers. In addition, drag on the hydraulic fluid flowing through the channels reduces efficiency, thereby requiring the pump to use additional energy in order to drive the fluid flow.

U.S. Pat. No. 5,306,295 by Kolff et al. was designed, in part, to overcome these problems in the art. Kolff eliminates the channels used by Jarvik by moving the energy converter between the blood pumping chambers. The energy converter includes an axial flow pump that communicates with the left and right pumping chambers. The position of the motor was designed to reduce the displacement distance required for the pumping fluid.

While the Kolff device was effective in reducing frictional drag by eliminating the conduits, it is not without drawbacks. In order to achieve the desired pump placement, Kolff must use a relatively inefficient, small-diameter energy converter with the impeller blades placed in the center of the motor proximate the rotational axis. The resulting low efficiency pump requires high RPM operation and a large amount of energy to drive the reciprocating fluid flow necessary for the blood pump's operation.

Accordingly, there exists a need for a low profile, efficient electrohydraulic energy converter for use in a circulatory assist device.

### SUMMARY OF THE INVENTION

The present invention provides a device for pumping blood that is particularly useful in a circulatory assist device. In one aspect, a circulatory assist device includes a blood pumping element having a first hemocompatible surface and a second fluid contacting surface, and a reversible axial flow pump. The reversible axial flow pump is in fluid communication with the blood pumping element and provides an annular fluid flow to and from the second fluid contacting surface of the blood pumping element to cause the first hemocompatible surface of the blood pumping element to expand into a blood supply and retract away from a blood supply to effect the pumping of blood. The various advantages of applying an annular fluid flow to a blood pumping element as well as configurations for providing such annular flow are described in detail below.

In another aspect, the elements of the invention are applied in a total artificial heart having left and right blood pumping chambers. Left and right flexible blood pumping elements are disposed in the total artificial heart so as to extend into and retract away from the left and right blood pumping chambers respectively for pumping blood therethrough. An axial flow pump is provided in the total artificial heart between the left and right blood pumping chambers for reversibly pumping hydraulic fluid between the left and right blood pumping elements. The axial flow pump is configured to provide annular fluid flow toward each of the left and right blood pumping elements.

In still another aspect of the invention, a blood pumping actuator for alternately driving left and right systole in a circulatory assist device. The actuator includes a housing having first and second open ends, and first and second blood pumping elements disposed on the first and second open ends of the housing. An axial flow pump capable of reversible flow is disposed within the housing. The axial flow pump has a substantially disk shaped motor and a plurality of rotor blades disposed on the motor and extending radially outward from the motor. The axial flow pump is configured to provide to provide annular flow of a hydraulic fluid reciprocally in directions toward the first and second blood pumping elements.

In a further aspect of the invention, a blood pump is provided for alternately driving left and right systole in a circulatory assist device. The blood pump includes a housing that defines a hydraulic fluid flow path and has first and second open ends. A reversible axial flow pump is disposed within the housing for reversibly pumping hydraulic fluid along the hydraulic fluid flow path. A left blood pumping element is disposed on and seals the first open end of the housing and a right blood pumping element is disposed on and seals the second open end of the housing. The axial flow pump causes hydraulic fluid flow in each of two directions, the first direction moving the hydraulic fluid toward the left blood pumping element and away from the right blood pumping element causing the left blood pumping element to extend and the right blood pumping element to retract, and the second direction moving the hydraulic fluid toward the right blood pumping element and away from the left blood pumping element causing the right blood pumping element to extend and the left blood pumping element to retract.

In certain embodiments of the invention, the reversible axial flow pump has a substantially disk shaped motor, a stator, a rotor rotatably connected to the stator, and several rotor blades disposed around and extending radially outward from the rotor. The rotor blades act against fluid to pump the fluid to and from the left and right blood pumping elements. The stator may contain several stator blades positioned on opposed sides of the impeller blades along the fluid flow path. The stator blades convert rotational fluid flow caused by the impeller blades into generally axial flow. Circumferential stator blades may also be employed.

In further embodiments of the invention, a motor is disposed inward of the rotor blades and imparts mechanical movement to the rotor. The motor includes, for example, a magnet disposed within the rotor and a coil disposed within the stator, which interact to impart mechanical movement on the rotor. The motor can be in electronic communication with a controller, which provides a reversing drive signal to the motor for reversible operation. The axial flow pump can also contain a pressure sensor for sensing the pressure at each blood pumping element. A stroke sensor may also be provided for sensing whether the left and/or right blood pumping elements have achieved full stroke.

In some embodiments, blood pumping elements are provided in the form of a dome or bowl shaped flexible diaphragm that flexes in two directions. The diaphragm can be mated to the end of a housing and flex above and below the end of the housing as a result of reversible flow from an axial flow pump, while not contacting the axial flow pump, even while flexing below the housing towards the pump. Blood pumping elements can also be provided as joined to a blood pumping chamber. Such a blood pumping element can be joined to the blood pumping chamber and mated to an inner surface thereof from the joining region to an apex of the blood pumping chamber so as to form a smooth, seamless, hemocompatible blood pumping chamber.

### BRIEF DESCRIPTION OF THE DRAWINGS

The invention will be more fully understood from the following detailed description taken in conjunction with the accompanying drawings, in which:

FIG. 1 is a perspective view of a blood pumping actuator having an axial flow pump in accordance with the present invention;

FIG. 2 is a diagrammatic view of a total artificial heart employing the blood pumping actuator of FIG. 1.

FIG. 3 is a cross-sectional view of the axial flow pump of the energy converter of FIG. 1;

FIG. 4 is a cross-sectional view of the axial flow pump of FIG. 3 taken along line 4-4;

FIG. 5 is an exploded view of the rotor and stator components of the axial flow pump of FIG. 2;

FIG. 6 is a cross-sectional view of the axial flow pump of FIG. 2 further comprising stator blades;

FIG. 7 is a diagrammatic view of rotor blades and stator blades of the axial flow pump of FIG. 6; FIG. 8 is a cross-sectional view of an axial flow pump of the invention having dome shaped surfaces;

FIG. 9 is a cross-sectional view of a blood pumping device of the invention employing the axial flow pump of FIG. 8;

FIGS. 10, 10A and 10B illustrate a top view and cross-sections of an axial flow pump of the invention having circumferential stator blades;

FIG. 11 is a cross-sectional view of a further axial flow pump of the invention;

FIGS. 12 and 12A through 12E are cross-sectional views illustrating the operation of a blood pumping chamber such as a blood pumping chamber employed by the blood pumping device of FIG. 9;

FIGS. 13 and 14 illustrate the operation of a further blood pumping chamber of the invention; and

FIG. 15 illustrates the flow of blood through a blood pumping chamber of the invention.

### DETAILED DESCRIPTION OF THE INVENTION

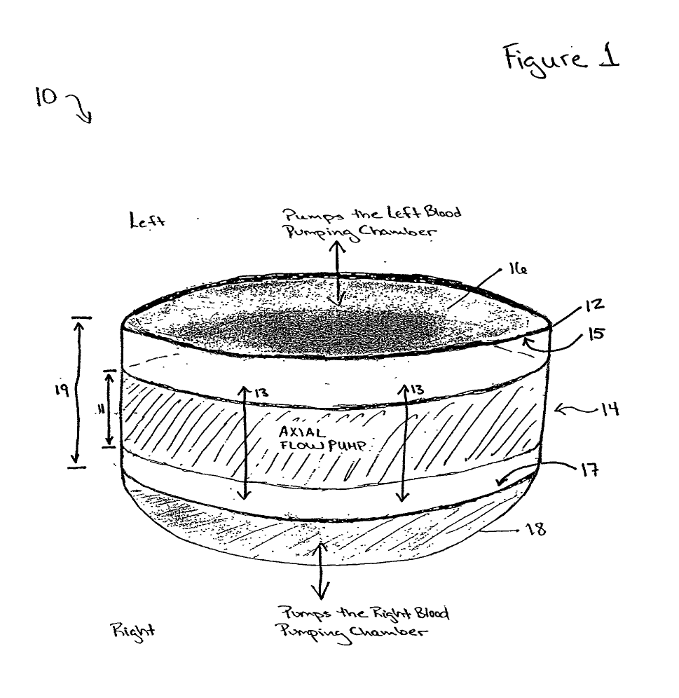

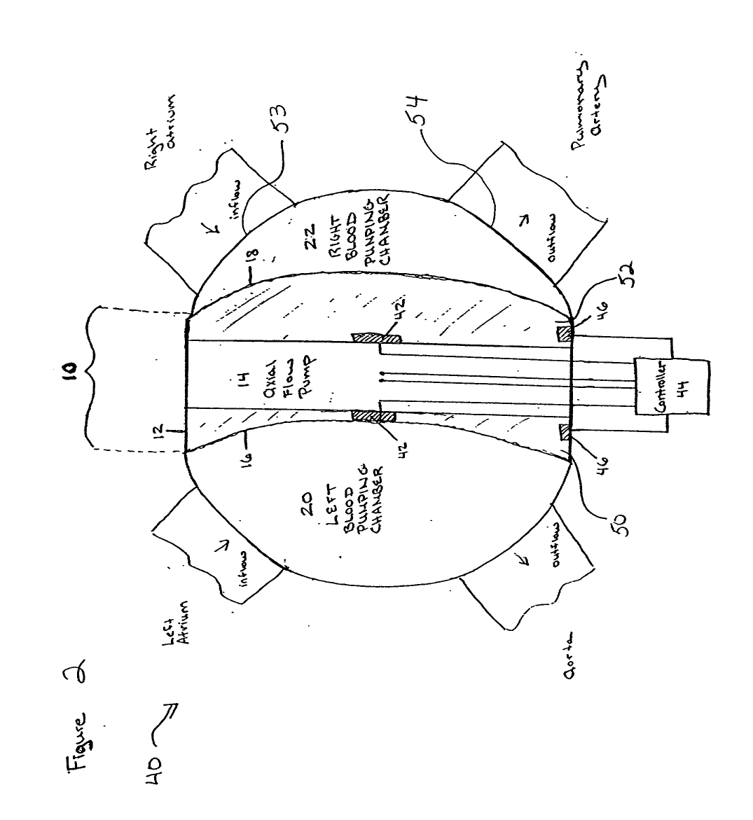

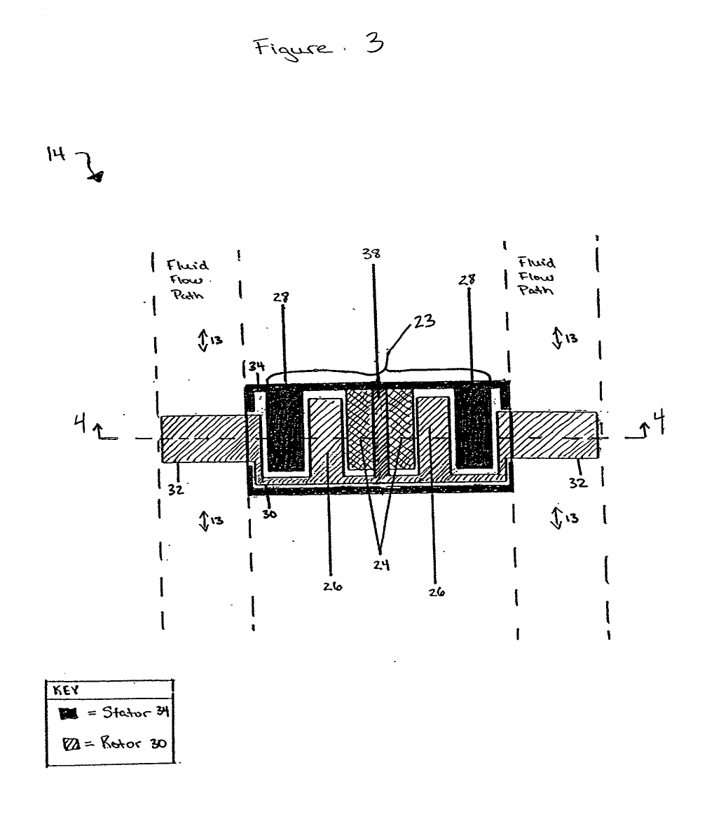

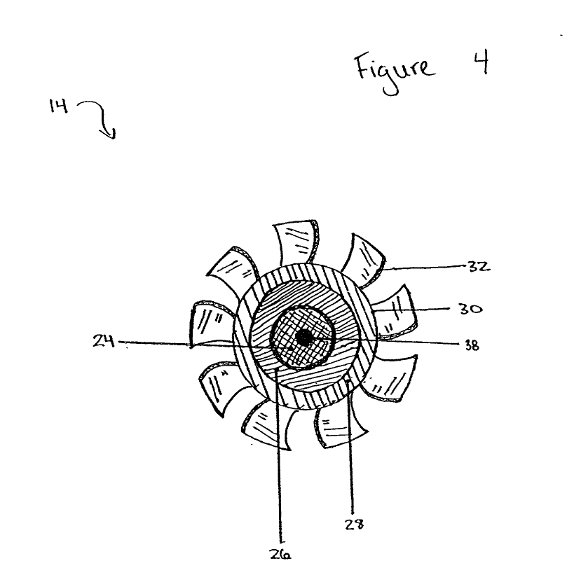

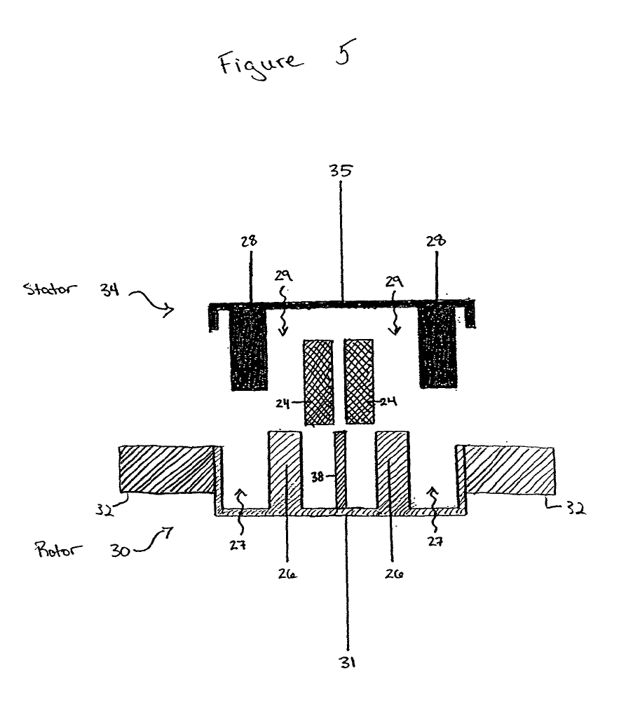

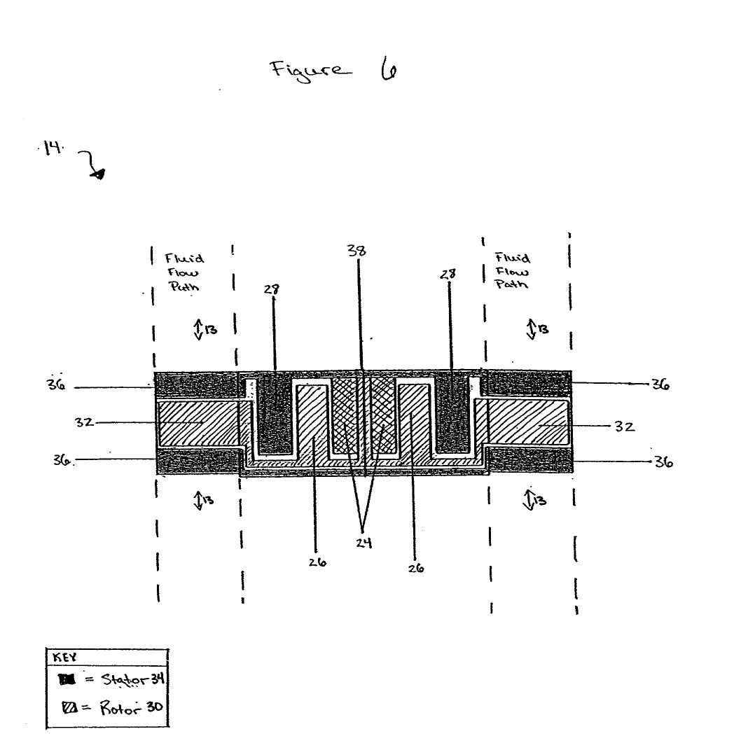

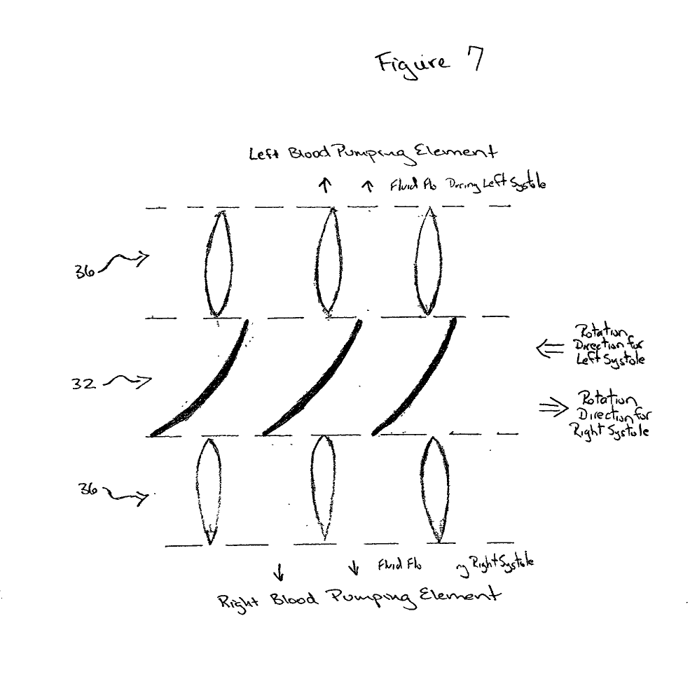

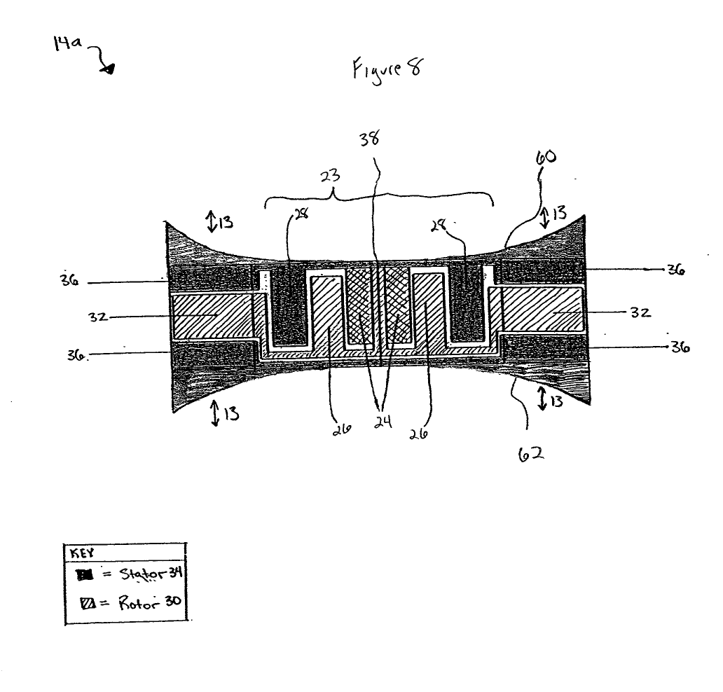

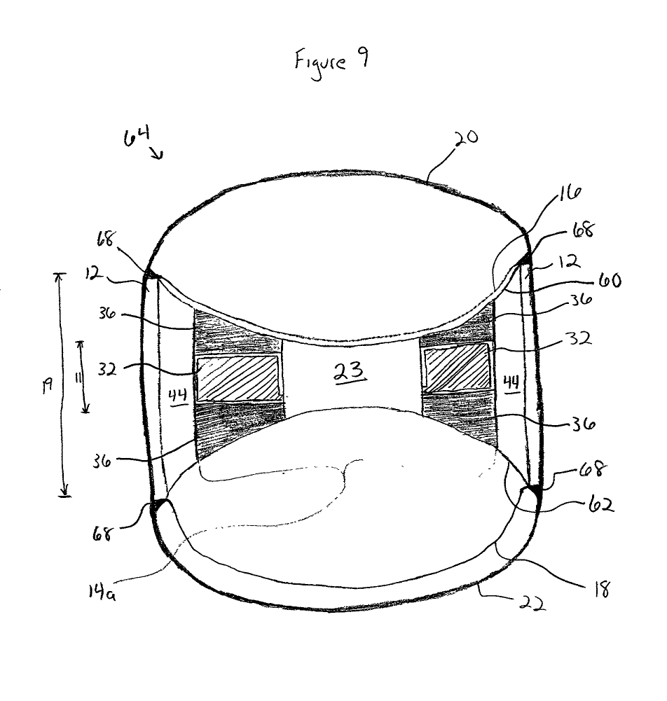

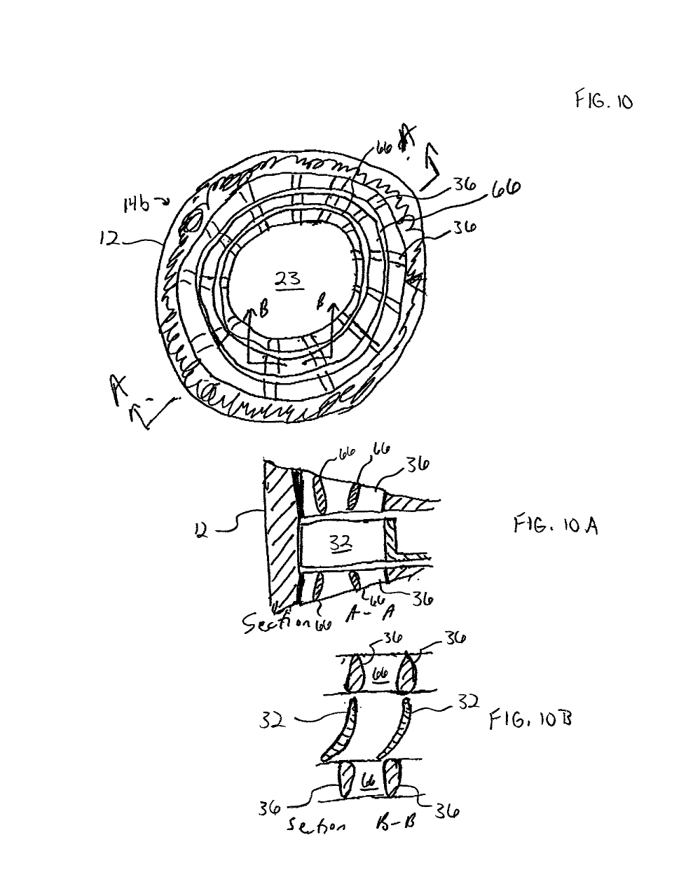

FIG. 1 illustrates a blood pumping actuator 10 in accordance with the present invention having a substantially cylindrical housing 12 having two open ends 15, 17. A left blood pumping element 16 and a right blood pumping element 18 are disposed on and seal the two open ends 15, 17 of housing 12. A reversible axial flow pump 14 is disposed within housing 12 to reversibly drive the flow of a hydraulic fluid along flow path 13.

Axial flow pump 14 reversibly pumps hydraulic fluid flow in two directions along fluid flow path 13. As shown in FIG. 1, pumping in a first direction moves hydraulic fluid toward right blood pumping element 18 and away from left blood pumping element 16, causing right blood pumping element 18 to extend and left blood pumping element 16 to retract. Pumping in a second direction moves hydraulic fluid toward left blood pumping element 16 and away from right blood pumping element 18, causing left blood pumping element 16 to extend and right blood pumping element 18 to retract.

When blood pumping actuator 10 is used in a biventricular cardiac prosthesis, left blood pumping element 16 is adapted to mate with a left blood pumping chamber (or artificial ventricle), which is then placed in fluid communication with the aorta and the left atrium. Right blood pumping element 18 is adapted to mate with a right blood pumping chamber (or artificial ventricle), which is then placed in fluid communication with the pulmonary artery and the right atrium.

FIG. 2 illustrates diagrammatically blood pumping actuator 10 of the present invention deployed in a biventricular cardiac prosthesis, namely Total Artificial Heart (TAH) 40. TAH 40 generally includes left and right blood pumping chambers 20, 22, with blood pumping actuator 10 disposed between left and right blood pumping chambers 20, 22. Blood pumping actuator 10 has left and right blood pumping elements 16, 18, and a reversible axial flow pump 14 for reversibly pumping hydraulic fluid to extend and retract left and right blood pumping elements 16, 18. Due at least in part to the low height of axial flow pump 14, TAH 40 is reduced in size compared to total artificial hearts known in the art.

Axial flow pump 14 can have at least one pressure sensor 46 for sensing the pressure in each fluid pumping chamber 50, 52. Axial flow pump 14 can also include at least one stoke sensor 42 for sensing whether left or right diaphragm 16, 18 has achieved a full stroke. A controller 44 can be used to communicate with motor 23, pressure sensor(s) 46, and stroke sensor(s) 42 to switch the power on and off to different windings of motor 23 at appropriate times to maintain an appropriate motor 23 speed and direction.

Controller 44 can also include motor control electronics having a fixed memory device can also be provided containing logic for recognizing the direction of rotation of motor 23, the motor speed and the torque the motor 23 is applying to axial flow pump 14. The motor control electronics command motor 23 to operate at a desired speed and to reverse at appropriate times. The motor control electronics may utilize feedback control from pressure sensors 46 in the hydraulic fluid or may recognize when the diaphragm has reached full extension by a build-up of pressure and the resulting change in the power requirements to motor 23. Additionally, the motor control electronics may receive external commands such as a signal from an electrocardiogram, or may refer to a stored data bank of information to determine the instantaneous motor direction and speed.

As shown in FIG. 2, when hydraulic fluid is pumped toward right blood pumping element 18, right blood pumping element 18 extends and forces blood out of right blood pumping chamber 22 and into the pulmonary artery. At the same time, axial flow pump 14 withdraws hydraulic fluid to cause left blood pumping element 16 to retract as blood flows into left blood pumping chamber 20 from the left atrium. Blood flow through left blood pumping chamber 20 is generally controlled by pressure sensitive valves 53, 54 as is known in the art. In addition, the retraction of left blood pumping element 16 can aid in drawing blood from the left atrium into left blood pumping chamber 20 by creating favorable pressure conditions for such blood flow. Once the stroke is complete, axial flow pump 14 reverses direction (not shown) and pumps hydraulic fluid toward left blood pumping element 16. Left blood pumping element 16 extends and forces blood out of left blood pumping chamber 20 into the aorta. Simultaneously, right blood pumping element 18 retracts as blood flows into the right blood pumping chamber 22 from the right atrium.

Each of left and right blood pumping elements 16, 18 can be a flexible or distensible diaphragm or membrane, or other pumping element known in the art. In one preferred embodiment, left and right blood pumping elements 16, 18 are flexible, substantially inelastic diaphragms of generally even thickness that are preformed in a dome or bowl shape. The material used for the left and right blood pumping elements 16, 18 should be durable and biocompatible. When used in a circulatory assist device, the materials used in blood pumping elements 16, 18 should be hemocompatible or coated with a hemocompatible material as they come into contact with the blood. That is, the materials should be non-mutagenic, non-toxic, and non-thrombogenic. Suitable biocompatible materials include, for example, medical grade silicones, epoxy, and polyetherurethane. One preferred material for blood contacting surfaces is ANGIOFLEX® available from ABIOMED, Inc. of Danvers, Mass.

Axial flow pump 14 can have a height 11 less than height 19 of housing 12, such that housing 12 extends beyond the axial flow pump toward each open end 15, 17. This extended height 19 of housing 12 permits left and right blood pumping elements 16, 18 to have a pre-formed bowl shape, as illustrated in FIG. 1. The use of a dome or bowl shaped pumping element 16, 18 helps prevent wrinkling and fluid flow blockage and helps the pumping elements to be manufactured in a single thickness. In addition, the bowl shape reduces side to side motion within the pumping element, thereby reducing wear on the pumping element. In one particular embodiment, housing 12 has a height 19 no greater than about 0.6 inches. In order to achieve these desired results, axial flow pump 14 must have a particularly low profile.

Further details of the axial flow pump 14 embodiment of FIG. 1 are shown in FIG. 3. Axial flow pump 14 includes a motor section 23 for imparting mechanical movement to a rotor 30 with respect to a stator 34. As used herein, the term rotor refers to the rotating portion of axial flow pump 14, including the rotating portion of motor 23 as well as rotor blades 32 which are connected to the rotating portion of the motor for rotation therewith. Motor section 23 can include, for example, a magnet 26 disposed on rotor 30, which interacts with a coil 28 disposed on stator 34. Motor 23 can have any shape, but is preferably substantially disk shaped. A suitable motor for use with axial flow pump 14 is a brushless DC motor, however, a person having ordinary skill in the art will readily appreciate that any motor suitable for the intended purpose of the invention can be used to drive axial flow pump 14. A brushless DC motor typically has a wound stator, a permanent magnet rotor assembly, and internal or external sensing devices to sense rotor position. The sensing devices produce signals for electronically switching (commutating) the stator windings in the proper sequence to maintain rotation of the magnet assembly.

Rotor blades 32 can be disposed around and extend radially outward from motor 23 portion of rotor 30. Rotor blades 32 act against the fluid along fluid flow path 13, as indicated by the dotted lines shown in FIG. 3. Placement of rotor blades 32 around the outer diameter of axial flow pump 14 increases the efficiency of the pump, at least in part because the weight of rotor 30 concentrated near its center with the result that rotational inertia is reduced and less energy is required to drive the reciprocating fluid flow necessary for the pump's operation. Placing rotor blades 32 around the outer diameter also allows for greater blade velocity at a lower rotational velocity for motor 23 than other configurations, thus requiring less energy to decelerate and accelerate axial flow pump 14 on reversals. In addition, disposing rotor blades 32 about the periphery of motor 23 (without providing elements for reattaching the fluid flow away from the axial flow pump 14) imparts an annular fluid flow 13 toward pumping elements 16, 18 (FIG. 1). The advantages provided by annular flow 13 in the systems of the invention are described in greater detail below.

Axial flow pump 14 is shown having rotor 30 and stator 34 components rotatably connected to each other. One or more bearing elements 24 can be used to suspend the rotor 30 with respect to the stator 34 thereby preventing friction causing contact between rotor 30, stator 34, and surrounding housing 12 (shown in FIG. 1). Bearing 24 can interact with rotating rotor 30 through a shaft 38 provided on rotor 30, though a person of ordinary skill in the art will recognize that other bearing arrangements can be employed while keeping within the spirit of the invention.

The bearing 24 or bearings used to support rotor 30 should accept axial thrust as well as radial load. Specific types of bearings that may be used include, for example, ball-bearings, roller bearings, hydrodynamic bearings, or simple sleeve bearings with special design and special materials to permit adequate durability under the high-speed and rapidly reversing cycle. The use of hydrodynamic bearings, for example, substantially eliminates wear within the system since the fluids of the hydrodynamic bearing prevents the surfaces of rotor 30 and stator 34 from touching.

FIG. 4 shows a cross-sectional view of the axial flow pump 14 of FIG. 3. Bearing 24 is disposed around shaft 38. Magnets 26 are disposed around bearing 24, and coil 28 is disposed around magnets 26. Rotor blades 32 are shown extending radially outward from rotor 30. In one embodiment, the outer diameter of the rotor blades 32 is approximately the same as, or only slightly smaller than, the diameter of the open ends of the housing 12 (FIG. 1). Rotor blades 32 may also be shrouded to minimize fluid flow in the region between the outer edge of the rotor blades and housing 12. In one particular embodiment, the inside diameter of rotor blades 32 can be approximately 3.0 inches and the blades can be approximately 0.25 inches wide. Rotor blades 32 can also be curved to be more efficient in pumping hydraulic fluid in a preferred direction along the fluid flow path. In the illustrated embodiment, rotor blades 32 are curved (best illustrated in FIG. 7) to favor flow in a direction toward a left blood pumping chamber (and thus disfavoring flow toward a right blood pumping chamber). This can be a desirable configuration in a biventricular cardiac prosthesis where blood pressure is typically higher on the left side of the heart, with the result that left blood pumping element 16 must do more work to pump blood than right blood pumping element 18.

As illustrated in the exploded view of FIG. 5, rotor 30 and stator 34 can interact to reduce the overall height 11 (FIG. 1) for the axial flow pump 14. Rotor 30 can include a shaft 38, a base 31, one or more magnets 26 arranged in an annular fashion extending from the base 31, and an annular groove 27 defined adjacent to the one or more magnets 26. The stator 34 can also include a base 35, one or more coils 28 arranged in an annular fashion extending from the base 35, and an annular groove 29 defined adjacent to the one or more coils 28. When the rotor 30 and stator 34 are joined together by a bearing 24, as illustrated in the embodiment of FIG. 4, the one or more magnets 26 on the rotor 30 extend into the annular groove 29 in the stator 34, and the one or more coils 28 on the stator 34 extend into the annular groove 27 of the rotor 30, thereby providing a reduced height 11 (FIG. 1) for the axial flow pump 14.

A further embodiment of the axial flow pump 14 of present invention is shown in FIG. 6. Several stator blades 36 are disposed about the periphery of stator 30, positioned on opposed sides of rotor blades 32 along the fluid flow path 13. As with the term rotor, as used herein, the term stator refers to the stationary portion of axial flow pump 14, including the stationary portion of motor 23 as well as stator blades 36. Stator blades 36 interact with rotor blades 32 to control the direction of fluid flow along the fluid flow path and to convert rotational fluid flow caused by the impeller blades 32 into generally annular axial flow.

FIG. 7 illustrates diagrammatically how stator blades 36 and rotor blades 32 can be configured to provide optimal annular fluid flow. For example, stator blades 36 can be wider in the center and decreasing in width in the direction of fluid exit (in the directions of left and right systole as noted in FIG. 7) along the fluid flow path so as to reattach the annular flow about stator blades 36 (and preferably not to reattach the annular flow into a full axial flow across the diameter of the axial flow pump). Stator blades 36 can be straight in order to straighten fluid flow exiting the axial flow pump that would otherwise include a circumferential component imparted to it by the rotating rotor blades 32. Stator blades 36 can also be angled to achieve a pressure differential between the left and right blood pumping chambers should such a pressure differential be desired, for example, to account for natural pressure differences between left and right chambers in the heart. Stator blades 36 can also be angled to optimally perform the function of accelerating and de-accelerating hydraulic fluid flow.

A reversible axial flow pump 14a is illustrated in FIG. 8 that is substantially similar to reversible axial flow pump 14 of FIG. 6, but having concave surfaces 60 and 62 in each of the directions of annular flow 13 from reversible axial flow pump 14a. The concave shape of the surfaces 60 and 62 impart a number of advantages to axial flow pump 14a that can be observed by reference to blood pumping device 64 of FIG. 9. Blood pumping device 64 is illustrated in FIG. 9 at the end of right systole with right blood pumping element 18 fully extended into right blood pumping chamber 22, and left blood pumping element 16 fully retracted from left blood pumping chamber 20 marking the start of left systole. As with blood pumping actuator 10 of FIG. 1, the retracted blood pumping element 16 is retracted to a height that is below the height 19 of circumferential housing 12, but which is not retracted to height 11 of axial flow pump 14, 14a— that is, the blood pumping element does not touch the axial flow pump. This configuration allows for optimal space utilization without subjecting blood pumping elements 16, 18 to the impact and frictional forces that would result if the blood pumping elements did contact axial flow pump 14, 14a. Concave surfaces 60, 62 can be designed in conjunction with the preformed shape of blood pumping elements 16, 18 to maximize this effect.

In addition, concave surfaces 60, 62 can contribute to optimal sizing of axial flow pump 14a. Stator blades 36 having increasing efficiency along with increased length in the flow direction. Concave surfaces 60, 62 allow for stator blades 36 to have a greater length in the desired flow direction near the circumference of the pump 14a in order to help counter the effect of reduced length of the stator blades closer to the motor 23. Accordingly, in particular where the height of axial flow pump 14a is limited by a necessary size of stator blades 36, the profile of axial pump 14a can be reduced, especially toward its center, by employing concave surfaces 60, 62 with correspondingly shaped stator blades 36.

Also significant in the configuration of blood pumping device 64 is the dome shape of the blood pumping chambers 20, 22. The dome shape of the blood pumping chambers 20, 22, combined with the dome shape of concave surfaces 60, 62, allows a similar dome or bowl shape for blood pumping elements 16, 18. This configuration allows optimal blood pumping volume for the size of blood pumping device 64 while maintaining a smaller angular movement of blood pumping elements 16, 18 at their connection 68 around the periphery of each blood pumping chamber 20, 22 during operation with equal flexing up and down (above and below height 19 of housing 12), resulting in greater longevity for the material used in the blood pumping elements.

Referring now to FIGS. 10, 10A and 10B, an axial flow pump 14b having circumferential 66 as well as radial 36 stator blades is illustrated. In addition to providing control over circumferential components of fluid flow using radial stator blades 36, control over radial flow caused by centrifugal action in flow pump 14b can be gained by providing one or more circumferential stator blades 66 as illustrated. Whether to employ a circumferential stator blade 66 depends upon the relative sizes and shapes of the components used. A further axial flow pump 14c of the invention is illustrated in FIG. 11. Axial flow pump 14c maintains concave surfaces 60 and 62 on the flow direction surfaces of the pump, but angles rotor blades 32 in a radial plane away from a preferred flow direction in order to provide a preferred flow direction 70 for the pump. Because of the angling of rotor blades 32, the flow from the blades will be driven not only by the axial flow component imparted on the fluid by the rotating rotor blades, but also a centrifugal component. This results in a stronger flow in the preferred direction 70 and a reduced flow in the direction opposite to the preferred direction. Depending on the configuration of a blood pumping device using axial flow pump 14c, it may be desirable to provide larger radial stator blades 36 on the side of the preferred flow direction 70, and perhaps also circumferential stator blades such as stator blades 66 (FIGS. 10, 10A, 10B) in order to straighten the annular axial flow provided by the pump.

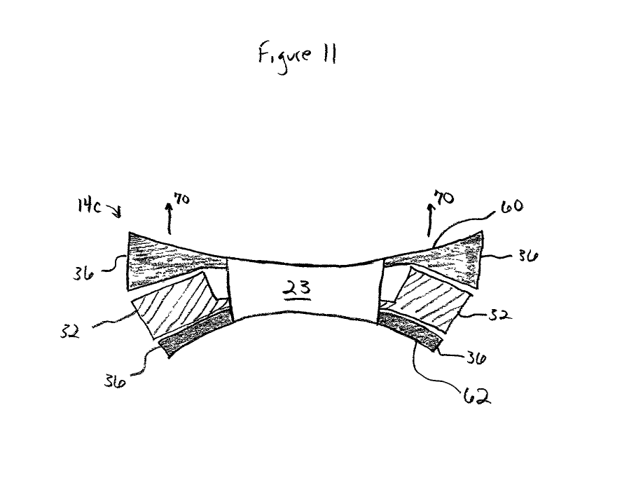

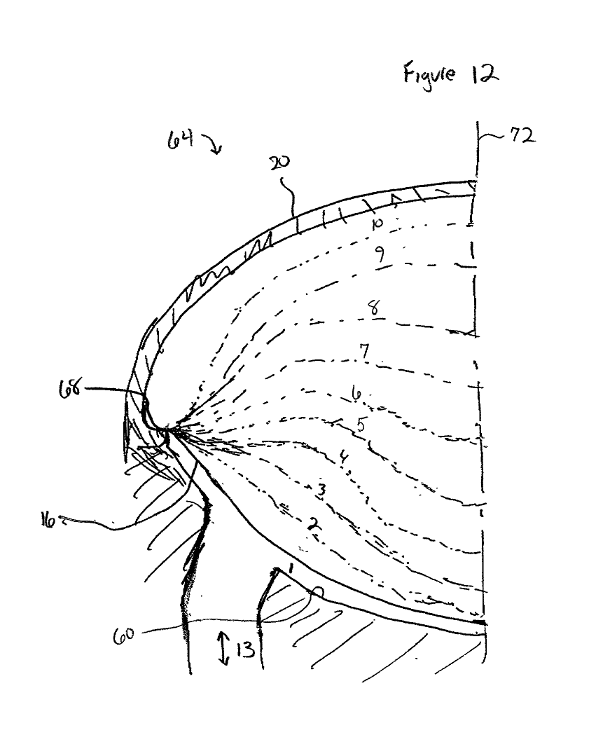

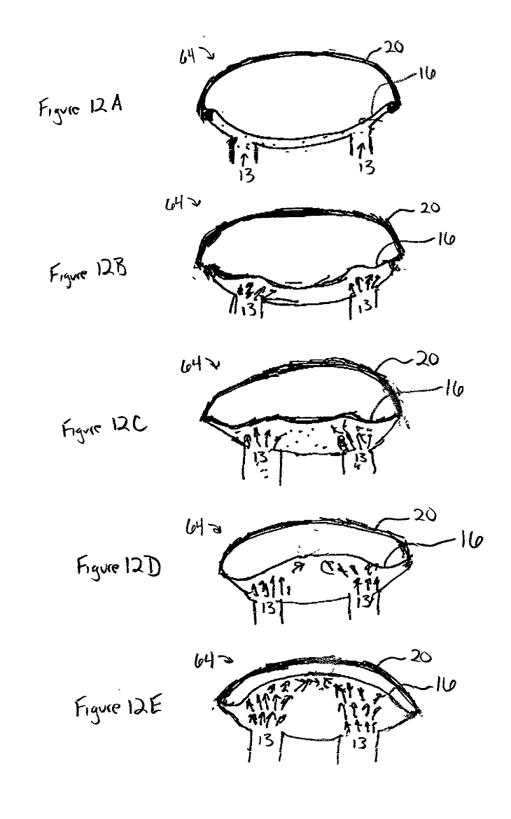

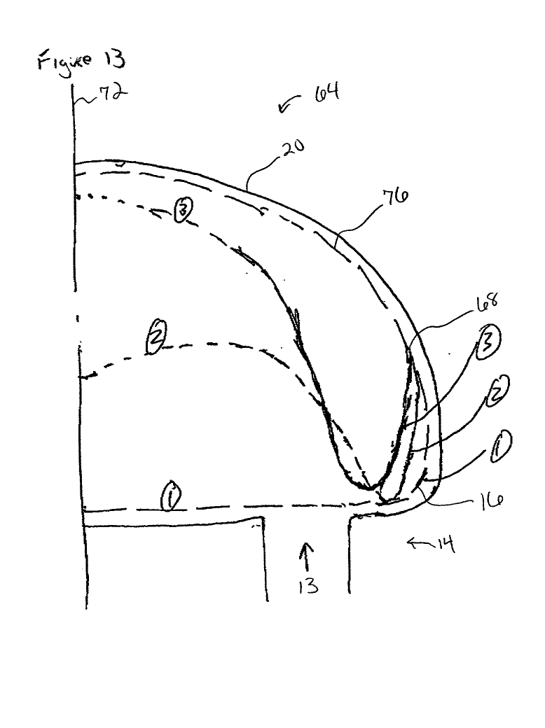

FIGS. 12 though 14 illustrate some of the benefits of the axial flow pump of the invention having annular flow using different blood pumping element configurations. In FIG. 12, one half of a blood pumping device 64 is illustrated in cross section. As blood pumping device 64 and annular flow 13 are generally axially symmetric about axis 72, the movement of dome or bowl shaped blood pumping element 16, connected to left blood pumping element 20 peripherally at connection region 68, will also be generally symmetric about axis 72. Blood pumping element 16 is illustrated in 10 successive positions numbered 1 (shown as solid), and 2 through 10 (shown ghosted). In position 1, the blood pumping element is illustrated at the start of left systole, with left blood pumping element 20 filled to capacity with blood to be pumped. Successive positions 2 through 10 of the blood pumping element illustrate its movement as hydraulic fluid is pumped in an annular pattern 13 so as to extend blood pumping element 16 to the end of left systole (position 10) where the maximum capacity of blood has been expelled from left blood pumping chamber 20. As the figure illustrates, blood pumping element 16 moves from full retraction (position 1) to full extension (position 10) without wrinkling, without side to side movement, and without contacting the surface of blood pumping element 20 or concave surface 60. Depending on the materials used for the various parts and the speed at which blood pumping element 16 cycles, each of these features can have a significant impact on the expected useful life of blood pumping element 16.

The shape of the dome or bowl shaped blood pumping element 16 of FIG. 12 is further illustrated during pumping in FIGS. 12A through 12E which illustrate left blood pumping element 64 in cross section in successive stages from the beginning of left systole (FIG. 12A) to the end left systole (FIG. 12E). At each stage, blood pumping element 16 is smooth, unwrinkled and axially symmetric in shape.

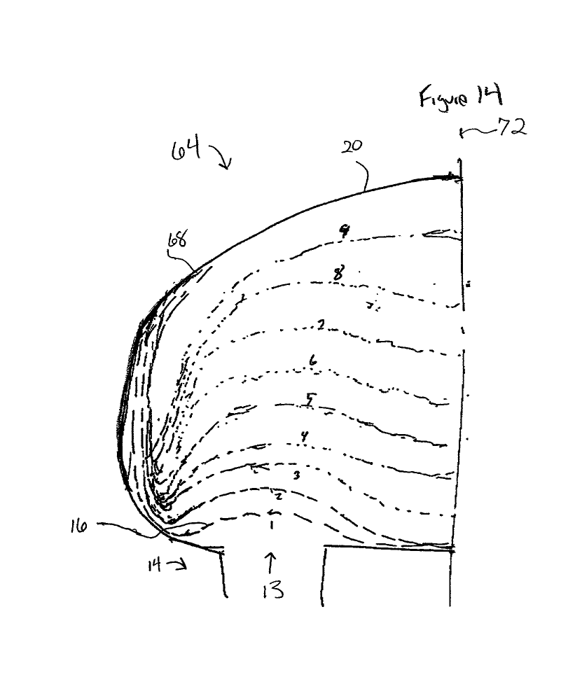

FIG. 13 illustrates a blood pumping device 64 having a disc-shaped annular axial flow pump 14 such as flow pump 14 of FIG. 6. Blood pumping element 16 is formed to the shape of the interior surfaces of left blood pumping chamber 20. In fact, it can be beneficial to form blood pumping element 16 from a hemocompatible substance that can be used to cover the entirety of an inner surface 76 of blood pumping chamber 20 so that the entire blood pumping chamber, including blood pumping element 16, is defined by a single, seamless, hemocompatible surface, allowing of course for blood entry and exit ports. Rather than being joined to the blood pumping chamber 20 proximate to the blood pumping chamber's connection to axial flow pump 14 as in earlier embodiments, blood pumping element 16 of FIG. 13 is joined to region 68 in the blood pumping chamber that is spaced apart from the axial flow pump 14. Where blood pumping element 16 forms an entire inner surface 76 of blood pumping chamber 20, the blood pumping element material can be bonded to inner surface 76 from an apex of the blood pumping chamber 20 along axis 72 down to joining region 68, allowing blood pumping element 16 to move freely below joining region 68. As can be seen from the numbered positions of blood pumping element 16 (position 1 representing the beginning of left systole through position 3 representing the end of left systole), in response to annular flow 13 the blood pumping element changes shape smoothly to pump blood from pumping chamber 20 without wrinkling or side to side movement of the blood pumping element.

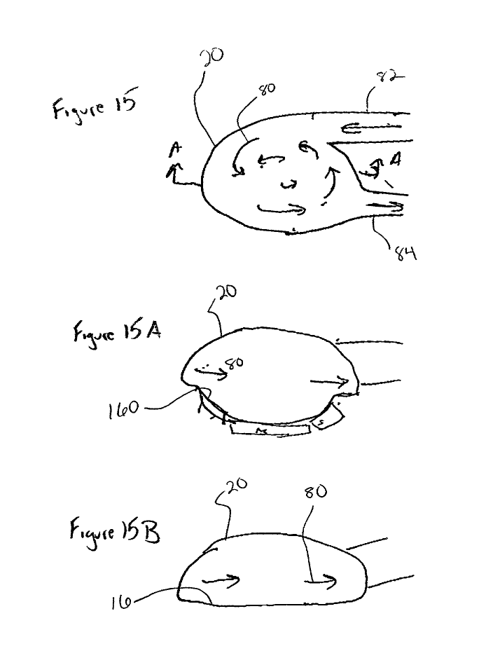

An additional configuration for blood pumping device 64 is illustrated in FIG. 14 wherein blood pumping element 16 is joined to left blood pumping chamber 20 at a position 68 that is closer to the apex of the blood pumping chamber at axis 72 than illustrated in FIG. 13. As shown by the 9 illustrated positions of blood pumping element 16, even in this configuration, the motion of blood pumping element 16 is symmetric and without wrinkling or side to side motion.

In a further configuration, blood pumping element 16 can be joined to the left blood pumping chamber 20 only at its inflow and outflow ports. In this way, blood pumping element 16 fills with blood, in a fashion similar to a balloon, through its inflow port during diastole, and empties through the outflow port under pressure from hydraulic fluid pumped by the axial flow pump during systole. During this blood pumping process, blood pumping membrane 20 expands and retracts in a manner similar to that shown in FIG. 14 wherein the blood pumping membrane is affixed blood pumping chamber 20 near its apex.

FIG. 15 provides a top view of a left blood pumping chamber 20 of the invention with blood flow through the chamber illustrated by arrows 80, and with blood inflow 82 and outflow 84 ports. Owing to steric constraints within the body, and also for hemocompatibility purposes, blood inflow port 82 is often placed so that the flow within the port is tangential to flow within the typically round blood pumping chamber 20. The speed of blood flow 80 within the blood pumping chamber 20 will be highest in proximity to the port in use for blood flow (inflow port 82 during diastole and outflow port 84 during systole), with the corresponding effect that pressure on the blood contacting side of a blood pumping element within the blood pumping chamber will be the lowest in these port regions. Where the blood pumping element is flexible, these low pressure regions on the blood contacting side can cause unevenness and even wrinkling in the blood pumping element. This tends to close off the port in use at the very time when it is called upon to sustain a high blood flow rate. Such an effect can be detrimental to blood flow within blood pumping chamber 20, to the amount of power required to pump the blood and to the longevity of the blood pumping element. A further advantage of the axial flow pumps of the invention having annular flow is that the annular flow is configured to counteract the low pressure regions that can form on the blood contacting side of a blood contacting element proximate to its periphery so as to maintain a uniformity in the blood pumping element that is beneficial for blood flow in the blood pumping chamber, for reducing the amount of power required to pump blood and for longevity of the blood pumping element.

FIGS. 15A and 15B illustrate blood flow 80 in cross-sectional views of the blood pumping chamber 20 of FIG. 15 at the end of diastole for configurations where an axial flow pump (such as axial flow pump 14a of FIG. 8) having a concave surface 60 is mated to blood pumping chamber 20 (FIG. 15A), and where a disc shaped axial flow pump (such as axial flow pump 14 of FIG. 6) is mated to blood pumping chamber 20 (FIG. 15B). As can be seen in FIGS. 15A and 15B, at diastole, blood can readily swirl throughout the entire blood pumping chamber, leaving no pockets of blood that can encourage thrombogenesis or hemostasis, and washing all of the blood contacting surfaces with blood flow even during low flow conditions.

While certain preferred embodiments have been disclosed above, the tenets of the invention can be applied to other embodiments as well. For example, blood pumping elements other than those described can be employed, such as elastic membranes and, while they may not make use of all of the advantages of the invention, rigid volume displacement elements such as pistons. In a further use of the elements of the invention, the axial flow pumps disclosed herein can be used within a ventricular assist device having only one blood pumping chamber. In such a system, the axial flow pump drives hydraulic fluid flow toward a blood pumping element in a first direct while directing hydraulic fluid flow toward a compliance chamber in a second direction rather than to a second blood pumping element.

One of ordinary skill in the art will know, or be able to ascertain, using no more than routine experimentation, many equivalents to the specific embodiments of the invention described herein. These and all other equivalents are intended to be encompassed by the following claims. All publications and references cited herein are expressly incorporated herein by reference in their entirety.

What is claimed is:

1. A blood pump for alternately driving left and right systole in a circulatory assist device, comprising:
a housing defining a hydraulic fluid flow path and having first and second open ends;
a reversible axial flow pump disposed within the housing for reversibly pumping hydraulic fluid along the hydraulic fluid flow path;
a left blood pumping element disposed on and sealing the first open end of the housing; and
a right blood pumping element disposed on and sealing the second open end of the housing;
wherein the axial flow pump causes hydraulic fluid flow in each of two directions, the first direction moving the hydraulic fluid toward the left blood pumping element and away from the right blood pumping element causing the left blood pumping element to extend and the right blood pumping element to retract, and the second direction moving the hydraulic fluid toward the right blood pumping element and away from the left blood pumping element causing the right blood pumping element to extend and the left blood pumping element to retract.

2. The blood pump of claim 1, wherein the axial flow pump comprises:
a stator;
a rotor rotatably connected to the stator, the rotor having a periphery and plurality of rotor blades arranged about the periphery of the rotor.

3. The blood pump of claim 2, wherein the axial flow pump having peripheral rotor blades is configured to provided annular flow in each of the two directions.

4. The blood pump of claim 3, further comprising a plurality of stator blades disposed on the stator, the stator blades being positioned on opposed sides of the rotor blades along the fluid flow path.

5. The blood pump of claim 4, further comprising at least one circumferential stator blade operatively connected to the plurality of stator blades disposed on the stator.

6. The blood pump of claim 2, wherein the blood pump further comprises:
a motor disposed inward of the rotor blades which imparts mechanical movement to the rotor.

7. The blood pump of claim 6, further comprising a controller in electronic communication with the motor and providing a reversing drive signal to the motor for reversible operation.

8. The blood pump of claim 7, further comprising a pressure sensor for sensing the pressure proximate to each blood pumping element, and further including stroke sensors for sensing whether the at least one of the left or right pumping elements has achieved a full stroke.

9. The blood pump of claim 1, wherein the housing is substantially cylindrical.

10. The blood pump of claim 9, wherein the substantially cylindrical housing has a height no greater than about 0.6 inches.

11. The blood pump of claim 1, wherein the left and right blood pumping elements each comprise a flexible diaphragm.

12. The blood pump of claim 11, wherein each diaphragm has a preformed bowl shape.

13. The blood pump of claim 11, wherein each diaphragm is elastic.

14. The blood pump of claim 11, wherein each diaphragm is inelastic and distensible.

15. The blood pump of claim 1, wherein the axial flow pump and the housing each have a height, the height of the axial flow pump being less than the height of the housing, wherein the pump and housing are configured so that the housing extends beyond the axial flow pump toward the left and right blood pumping elements.

16. The blood pump of claim 15, wherein each blood pumping element extends beyond the height of the housing, and retracts to a height below the height of the housing.

17. The blood pump of claim 16, wherein each blood pumping element is configured so as not to contact the axial flow pump upon retraction.

18. The blood pump of claim 2, wherein the rotor comprises a base, one or more magnets arranged in an annular fashion extending from the base, and an annular groove defined adjacent to the one or more magnets;
wherein the stator comprises a base, one or more coils arranged in an annular fashion extending from the base, and an annular groove defined adjacent to the one or more coils; and
wherein the rotor and stator are configured so that the one or more magnets extend into the annular groove in the stator and the one or more coils extend into the annular groove of the rotor to result in a reduced overall height for the axial flow pump.

19. The blood pump of claim 2, wherein the rotor blades are configured to provide a preferred direction of fluid flow.

20. A blood pumping actuator for alternately driving left and right systole in a circulatory assist device, comprising:
a housing having first and second open ends;
a first blood pumping element disposed on the first open end of the housing;
a second blood pumping element disposed on the second open end of the housing; and
an axial flow pump capable of reversible flow disposed within the housing, the axial flow pump having a substantially disk shaped motor and a plurality of rotor blades disposed thereon and extending radially outward therefrom, the axial flow pump configured to provide to provide annular flow of a hydraulic fluid reciprocally in directions toward the first and second blood pumping elements.

21. The blood pumping actuator of claim 20, further comprising a plurality of stator blades disposed on the axial flow pump, the stator blades being positioned downstream of the rotor blades along at least one direction along the fluid flow path.

22. The blood pumping actuator of claim 20, wherein the disk shaped motor further comprises:
a magnet disposed on the rotor; and
a coil disposed on the stator;
wherein the magnet and the coil interact to impart mechanical movement on the rotor.

23. The blood pumping actuator of claim 20, wherein the housing is substantially cylindrical and the blood pumping actuator is substantially disk shaped.

24. The blood pumping actuator of claim 20, wherein each blood pumping element is a diaphragm having a pre-formed bowl shape.

25. The blood pumping actuator of claim 20, wherein each blood pumping element is elastic.

26. The blood pumping actuator of claim 20, wherein each blood pumping element is inelastic and distensible.

27. The blood pumping actuator of claim 23, wherein the axial flow pump and the housing each have a height, the height of the axial flow pump being less than the height of the housing, wherein the pump and housing are configured so that the housing extends beyond the axial flow pump toward the first and second blood pumping elements.

28. The blood pumping actuator of claim 27, wherein the first and second blood pumping elements each expand beyond the height of the housing, and retract below the height of the housing.

29. A total artificial heart, comprising:
left and right blood pumping chambers;
left and right flexible blood pumping elements disposed so as to extend into and retract away from the left and right blood pumping chambers respectively for pumping blood therethrough; and
an axial flow pump disposed between the left and right blood pumping chambers for reversibly pumping hydraulic fluid between the left and right blood pumping elements, the axial flow pump configured to provide annular fluid flow toward each of the left and right blood pumping elements.

30. The total artificial heart of claim 29, wherein the left and right blood pumping chambers are each formed of a generally rigid outer shell, the left and right blood pumping elements being joined to the outer shell of the left and right blood pumping chambers respectively.

31. The total artificial heart of claim 30, wherein each of the left and right blood pumping elements expand beyond the region in which they are joined to their respective blood pumping chambers, and retract below the region in which they are joined to their respective blood pumping chambers in response to the flow of hydraulic fluid provided by the axial flow pump.

32. The total artificial heart of claim 31, wherein each of the left and right blood pumping elements expand and retract without wrinkling.

33. The total artificial heart of claim 32, wherein each of the left and right blood pumping elements is distensible and substantially inelastic.

34. The total artificial heart of claim 31, further comprising an inlet and outlet port included in each blood pumping chamber.

35. The total artificial heart of claim 34, wherein each of the left and right blood pumping elements is formed of a flexible material having a hemocompatible surface.

36. The total artificial heart of claim 35, wherein each of the left and right blood pumping elements is mated to their respective blood pumping chamber from the region in which they are joined to an apex of the respective blood pumping chamber to form a seamless hemocompatible surface for the respective blood pumping chamber while allowing for inlet and output ports.

37. The total artificial heart of claim 29, wherein the axial flow pump comprises:
a stator; and
a rotor rotatably connected to the stator and having a plurality of rotor blades disposed around a periphery of the rotor.

38. The total artificial heart of claim 37, further comprising a plurality of stator blades disposed on the stator positioned downstream from the rotor blades along at least one direction of the fluid flow path.

39. The total artificial heart of claim 37, wherein the axial flow pump further comprises:
a motor disposed inward of the peripheral rotor blades which imparts mechanical movement to the rotor with respect to the stator.

40. The total artificial heart of claim 39, further comprising a controller in electronic communication with the motor and providing a reversing drive signal to the motor for reversible operation.

41. The total artificial heart of claim 40, further comprising a pressure sensor in communication with the controller for sensing the pressure proximate to each blood pumping element, and further including stroke sensors for sensing whether the at least one of the left or right blood pumping element has achieved a full stroke.

42. The total artificial heart of claim 29, wherein the left and right blood pumping elements each comprise a flexible diaphragm.

43. The total artificial heart of claim 42, wherein each diaphragm has a pre-formed bowl shape.

44. The total artificial heart of claim 42, wherein each diaphragm is elastic.

45. The total artificial heart of claim 42, further comprising a housing disposed between the left and right blood pumping chambers and around the axial flow pump, the axial flow pump and the housing each having a height, the height of the axial flow pump being less than the height of the housing, wherein the axial flow pump and housing are configured so that the housing extends beyond the axial flow pump toward the left and right blood pumping chambers.

46. The total artificial heart of claim 45, wherein each blood pumping element extends beyond the height of the housing, and retracts to a height below the height of the housing.

47. The total artificial heart of claim 46, wherein each blood pumping element is configured so as not to contact the axial flow pump upon retraction.

48. The total artificial heart of claim 37, wherein the rotor comprises a base, one or more magnets arranged in an annular fashion extending from the base, and an annular groove defined adjacent to the one or more magnets;
wherein the stator comprises a base, one or more coils arranged in an annular fashion extending from the base, and an annular groove defined adjacent to the one or more coils; and
wherein the rotor and stator are configured so that the one or more magnets extend into the annular groove in the stator and the one or more coils extend into the annular groove of the rotor to result in a reduced overall height for the axial flow pump and reduced size for the total artificial heart.

49. The total artificial heart of claim 38, wherein the plurality of stator blades includes one or more circumferential stator blades.

50. A circulatory assist device comprising:
a blood pumping element having a first hemocompatible surface and a second fluid contacting surface; and
a reversible axial flow pump in fluid communication with the blood pumping element, the reversible axial flow pump providing an annular fluid flow to and from the second fluid contacting surface of the blood pumping element to cause the first hemocompatible surface of the blood pumping element to expand into a blood supply and retract away from a blood supply to effect the pumping of blood.

51. The circulatory assist device of claim 50, further comprising a housing having an open end, the blood pumping element being disposed on and sealing the open end.

52. The circulatory assist device of claim 50, wherein the axial flow pump comprises:
a stator;
a rotor rotatably connected to the stator, the rotor having a periphery and plurality of rotor blades arranged about the periphery of the rotor.

53. The circulatory assist device of claim 52, further comprising a plurality of stator blades disposed on the stator, the stator blades being positioned adjacent to the rotor blades along the fluid flow path toward the blood pumping element.

54. The circulatory assist device of claim 53, further comprising at least one circumferential stator blade operatively connected to the plurality of stator blades disposed on the stator.

55. The circulatory assist device of claim 52, wherein the circulatory assist device further comprises:
a motor disposed inward of the rotor blades which imparts mechanical movement to the rotor.

56. The circulatory assist device of claim 55, further comprising a controller in electronic communication with the motor and providing a reversing drive signal to the motor for reversible operation.

57. The circulatory assist device of claim 56, further comprising a pressure sensor for sensing the pressure proximate to the blood pumping element, and further including a stroke sensor for sensing whether the blood pumping element has achieved a full stroke.

58. The circulatory assist device of claim 51, wherein the housing is substantially cylindrical.

59. The circulatory assist device of claim 58, wherein the substantially cylindrical housing has a height no greater than about 0.6 inches.

60. The circulatory assist device of claim 50, wherein the blood pumping element comprises a flexible diaphragm.

61. The circulatory assist device of claim 60, wherein the diaphragm has a preformed bowl shape.

62. The circulatory assist device of claim 60, wherein the diaphragm is elastic.

63. The circulatory assist device of claim 60, wherein the diaphragm is inelastic and distensible.

64. The circulatory assist device of claim 51, wherein the axial flow pump and the housing each have a height, the height of the axial flow pump being less than the height of the housing, wherein the pump and housing are configured so that the housing extends beyond the axial flow pump toward the pumping element.

65. The circulatory assist device of claim 64, wherein the blood pumping element extends beyond the height of the housing, and retracts to a height below the height of the housing.

66. The circulatory assist device of claim 65, wherein the blood pumping element is configured so as not to contact the axial flow pump upon retraction.

67. The circulatory assist device of claim 52, wherein the rotor comprises a base, one or more magnets arranged in an annular fashion extending from the base, and an annular groove defined adjacent to the one or more magnets;
wherein the stator comprises a base, one or more coils arranged in an annular fashion extending from the base, and an annular groove defined adjacent to the one or more coils; and
wherein the rotor and stator are configured so that the one or more magnets extend into the annular groove in the stator and the one or more coils extend into the annular groove of the rotor to result in a reduced overall height for the axial flow pump.

68. The circulatory assist device of claim 52, wherein the rotor blades are configured to provide a preferred direction of fluid flow.

69. The circulatory assist device of claim 50, further comprising 29 a blood pumping chamber formed of a generally rigid outer shell.

70. The circulatory assist device of claim 69, wherein the blood pumping element is joined to an inner surface of the outer shell of the blood pumping chamber.

71. The circulatory assist device of claim 70, wherein the blood pumping element is joined to the blood pumping chamber on its second fluid contacting surface.

72. The circulatory assist device of claim 70, wherein the blood pumping element expands beyond the region in which it is joined to the blood pumping chamber, and retracts below the region in which it is joined to the blood pumping chamber in response to the flow of hydraulic fluid provided by the axial flow pump.

73. The circulatory assist device of claim 72, wherein each the blood pumping element expands and retracts without wrinkling.

74. The circulatory assist device of claim 73, wherein the pumping element is distensible and substantially inelastic.

75. The circulatory assist device of claim 72, further comprising an inlet and outlet port included in each blood pumping chamber.

76. The circulatory assist device of claim 75, wherein the blood pumping element is mated to the blood pumping chamber from the region in which they are joined to an apex of the blood pumping chamber to form a seamless hemocompatible surface for the respective blood pumping chamber while allowing for inlet and output ports.

77. The circulatory assist device of claim 50, further comprising a second blood pumping element in fluid communication with the reversible axial flow pump and disposed in an opposed relationship with the axial flow pump from the blood pumping element.

78. The circulatory assist device of claim 50, further comprising a compliance chamber in fluid communication with the reversible axial flow pump and disposed in an opposed relationship with the axial flow pump from the blood pumping element.
